(19) United States
(12) Patent Application Publication
(10) Pub. No.: US 2019/0289812 A1
(43) Pub. Date: Sep. 26, 2019

(54) ANIMAL LITTER WITH ODOR CONTROLLING COMPOUNDS

(71) Applicant: NESTEC SA, Vevey (CH)

(72) Inventor: James Li, Frontenac, MO (US)

(21) Appl. No.: 16/357,555

(22) Filed: Mar. 19, 2019

Related U.S. Application Data
(60) Provisional application No. 62/645,384, filed on Mar. 20, 2018.

(51) Int. Cl. A01K 1/015; B01J 20/12; B01J 20/10; B01J 20/28; A61L 2/20

(52) U.S. Cl. CPC A01K 1/0154; B01J 20/12; B01J 20/106; A61L 2/20; B01J 20/28016; B01J 20/28011; B01J 20/28059

(57) ABSTRACT

The present disclosure provides a composition for controlling malodor of animal waste, wherein the composition comprises a substrate with a specific surface area less than about 60 m²/g and an ammonia-controlling effective amount of a salt of chlorous acid and may further comprise an acid activating agent.

### CROSS REFERENCE TO RELATED APPLICATIONS

This application claims priority to U.S. Provisional Application Ser. No. 62/645,384 filed Mar. 20, 2018, the disclosure of which is incorporated in its entirety herein by this reference.

### FIELD

The present disclosure relates generally to absorbent granules containing odor controlling compounds that can be used in clumping pet litter. More specifically, the present disclosure is directed to absorbent granules comprising ammonia-controlling compounds.

### BACKGROUND

Litter boxes are used by pets such as cats for elimination of urine and fecal matter. A litter box contains a layer of pet litter that receives the urine and fecal matter. The pet litter is granular, absorbent, and either non-clumping or clumping. A clumping pet litter is a litter product in which the granules facilitate formation of clumps after the urine and fecal matter is deposited in the pet litter. The clumps are typically sifted from the litter box using a litter scoop and then discarded. Non-clumping pet litter is typically good at absorbing urine and thus removing urine odors, but replacing soiled non-clumping pet litter without emptying the entire box of litter can be difficult.

Animal waste, such as cat urine, contains a significant amount of urea. Urea decomposition is accelerated by an enzyme called urease, produced by microbes in the litter or surrounding environment. As a result of the enzymatic activity, ammonia will be generated, creating unpleasant and pungent ammonia odors. Therefore, ammonia control is critical in odor control of animal waste. In addition, the high humidity inside the urine/fecal clumps provides an ideal environment for microbes such as bacteria or viruses to grow.

There have been some studies in the area of ammonia control in various fields, including agriculture, medicine, human hygiene, and animal waste. For example, U.S. Pat No. 3,892,846 discloses a method of ammonia control using acetohydroxamic acid. Also, EP Pat. Nos. 0119487 and 0408199 disclose methods of controlling ammonia using phosphoric triamide and phosphorodiamide compounds, respectively. Additionally, U.S. Pat. No. 5,135,743 discloses an ammonia controlling combination comprising boric acid and pine oil. Each of these methods has advantages and disadvantages. There is therefore a need to further improve the performance of ammonia control. It is also desirable to counteract the malodors from microbe activities and maintain a clean and healthy environment surrounding the litter box in a cat owner's home.

### SUMMARY

The present inventors surprisingly found that odor control performance of absorbent granules used in clumping pet litter could be improved by incorporating an ammonia-controlling compound.

In one embodiment, the present disclosure provides a composition for controlling malodor of animal waste, comprising a substrate with a specific surface area of less than about 60 m²/g and a salt of chlorous acid in an amount from about 0.1 to about 10% by weight.

In another embodiment, the present disclosure provides a method of reducing malodor from animal waste comprising contacting the malodor with a composition comprising a substrate with a specific surface area of less than about 60 m²/g and a salt of chlorous acid in an amount from about 0.1% by weight to about 10% by weight.

In another embodiment, the present disclosure provides a method of reducing malodor from animal waste, the method comprising adding a deodorizer to a pet litter in a litter box, the pet litter having a different formulation than the deodorizer, the deodorizer comprising a substrate with a specific surface area of less than about 60 m²/g and a salt of chlorous acid in an amount from about 0.1% by weight to about 10% by weight.

### BRIEF DESCRIPTION OF THE FIGURES

FIG. 1 is a series of graphs showing the pH of different substrates containing citric acid and the pH after mixing citric acid modified substrates with an equal amount of unmodified substrate.

FIG. 2 is a graph showing formation of ClO₂ over time upon wetting a combination of citric acid modified litter substrate B and sodium chlorite (1%) modified Litter B substrate.

FIG. 3 is a graph showing the formation of ClO₂ over time upon wetting a combination of citric acid modified litter substrate B and sodium chlorite (5%) modified litter substrate B.

FIG. 4 is a graph showing the formation of ClO₂ over time upon wetting a combination of citric acid modified litter substrate B and sodium chlorite (5%) modified litter substrate B.

FIG. 5 is a graph showing the concentration of ClO₂ generated over time upon wetting of modified litter substrate C at a fixed citric acid concentration (0.5%) and varying sodium chlorite concentration.

FIG. 6 is a graph showing the effect of sodium chlorite and citric acid addition in litter on ammonia formation.

### DETAILED DESCRIPTION

Definitions

Some definitions are provided hereinafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “a material” or “the material” includes two or more materials.

The words “comprise,” “comprises” and “comprising” are to be interpreted inclusively rather than exclusively. Likewise, the terms “include,” “including” and “or” should all be construed to be inclusive, unless such a construction is clearly prohibited from the context.

However, the compositions disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. “Consisting essentially of” means that the embodiment comprises more than 50 wt. % of the identified components, preferably at least 75 wt. % of the identified components, more preferably at least 85 wt. % of the identified components, most preferably at least 95 wt. % of the identified components, for example at least 99 wt. % of the identified components.

The term “and/or” used in the context of “X and/or Y” should be interpreted as “X,” or “Y,” or “both X and Y.” Similarly, “at least one of X or Y” should be interpreted as “X,” or “Y,” or “both X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

The terms “pet” and “animal” are used synonymously herein and mean any animal which can use a litter box, non-limiting examples of which include a cat, a dog, a rat, a ferret, a hamster, a rabbit, an iguana, a pig or a bird. The pet can be any suitable animal, and the present disclosure is not limited to a specific pet animal. The term “elimination” means urination and/or defecation by a pet.

As used herein, the term “litter” means any substance that can absorb animal urine and/or decrease odor from animal urine and/or feces. A “clumping litter” forms aggregates in the presence of moisture, the aggregates distinct from the other litter in the litter box. A “clumping agent” binds adjacent particles when wetted. A “non-clumping litter” does not form distinct aggregates. The term “deodorizer” means any substance that absorbs at least a portion of surrounding malodors.

The term “litter box” means any apparatus that can hold pet litter, for example a container with a bottom wall and one or more side walls, and/or any apparatus configured for litter to be positioned thereon, for example a mat or a grate. As a non-limiting example, a litter box may be a rectangular box having side walls that have a height of at least about six inches.

The term “mesh” is defined by the ASTM E-11 U.S.A. standard specification for sieves. As used herein, “size” of a particle refers to the length of the longest dimension of the particle.

The methods and devices and other advances disclosed herein are not limited to particular methodologies, protocols, and reagents because, as the skilled artisan will appreciate, they may vary. Further, the terminology used herein is for the purpose of describing particular embodiments only and does not limit the scope of that which is disclosed or claimed.

Unless defined otherwise, all technical and scientific terms, terms of art, and acronyms used herein have the meanings commonly understood by one of ordinary skill in the art in the field(s) of the present disclosure or in the field(s) where the term is used. Although any compositions, methods, articles of manufacture, or other means or materials similar or equivalent to those described herein can be used, the preferred devices, methods, articles of manufacture, or other means or materials are described herein.

The present disclosure describes methods and compositions for control of ammonia odor of animal waste using salts of chlorous acid, also known as chlorite compounds. In one embodiment, the chlorite compounds can be alkali metal chlorite compounds such as sodium or potassium chlorite. It was discovered that chlorite salts can effectively reduce the ammonia generated within animal litter. The chlorite salts can be directly mixed and/or impregnated into the animal litter material, or other porous substrates that act as carriers. Without being bound by a particular theory, it is believed that the elimination or reduction of ammonia odor occurs via an oxidation process.

In some embodiments, the litter can further comprise an acidic activator, which can react with chlorite compounds at high moisture levels or when the litter is wetted by pet urination. The reaction of the chlorite compound with the acid activator (e.g. citric acid) will generate chlorine dioxide, an efficient deodorant and antimicrobial compound. The concentration of the chlorite compound and the acidic activator can be controlled to provide malodor control not only inside litter box, but also malodor control and antimicrobial properties for the space around the litter box.

In some embodiments, the chlorite compound itself is an efficient urease inhibitor. In other embodiments, the composition further comprises an acidic activator that can react with the chlorite compound thus generating chlorine dioxide, which not only reduces or eliminates odors within animal litter, but also provides deodorizing and/or antimicrobial function to the space surrounding the litter box. Controlled release of chlorine dioxide when the material is wetted by pet urination or at very high moisture levels is an advantageous aspect of the present invention.

Chlorine dioxide is an effective disinfectant, even at low concentrations, and is widely used in water disinfection, personal care products, food and beverage production, farms and also has applications in the medical field (e.g. hospitals). The present disclosure comprises a method to modify animal litter with a chlorite compound and an acid activator. The resultant litter remains stable at ambient usage, and the chlorine dioxide is only generated when the pet litter is wetted, for example by pet urination, thereby providing a disinfection function to the litter and surrounding environment, and also reducing or eliminating the ammonia generation.

The modification of pet litter can occur at any step in the manufacturing process. In some embodiments, the chlorite salt or chlorous acid can be added in later steps of the manufacturing process. In one embodiment, the pet litter can be modified with a chlorite salt only, or one portion of the litter can be modified with chlorite salt while another portion can be separately modified with an acid activator and the two separately modified litters can be separately dried and then mixed together. Various types of inorganic or organic acids can be used as the acid activator. In some embodiments, the acid activator can be citric acid, phosphoric acid, hydrochloric acid, or malic acid. In one embodiment, the acid activator is citric acid.

In some embodiments, the chlorite compound and acid activator are separately dissolved in water, and separately applied to the litter composition during the manufacturing of the litter. In other embodiments, the separate solutions containing the chlorite compound and the acid activator are separately sprayed onto the pet litter granules towards the end of the manufacturing process. In some embodiments, the citric acid modified litter can be prepared by dry mixing the litter with citric acid powder.

Because the generation of chlorine dioxide is a result of the reaction between chlorite salt and acid activator, either reactant can be used to control the final concentration of chlorine dioxide in pet litter or surrounding environment. However, it may be more convenient to use more citric acid to provide an acidic environment, while using the chlorite salt as the limiting reactant to control the quantity of chlorine gas generated. In an ideal situation, sufficient citric acid is added to litter substrate so that the pH of the final mixture of acid-modified litter and chlorite-modified litter is below 5, preferably below 4, even more preferably, below 3.5. This will ensure an acidic environment when the mixture is wetted by pet urination or defecation, and a majority of the chlorite salt will react with the acid and act as limiting agent in formation of chlorine dioxide gas.

In one embodiment, the present disclosure comprises a composition for controlling malodor in an animal litter, wherein the composition comprises a substrate with a specific surface area of less than about 60 m²/g and an ammonia-controlling effective amount of a salt of chlorous acid in an amount from about 0.1 to about 10% by weight. The effect of the substrate matrix (e.g. surface area) is an important component of the present disclosure. In some embodiments, the present disclosure comprises a composition for controlling malodor in an animal litter, wherein the composition comprises a substrate with a specific surface area of less than about 50 m²/g, less than about 40 m²/g, less than about 30 m²/g, less than about 20 m²/g, or less than about 10 m²/g and a salt of chlorous acid in an amount from about 0.1% to about 10% by weight. In some embodiments, the substrate is an animal litter.

In one embodiment, the salt of chlorous acid is selected from the group consisting of a sodium salt, a magnesium salt, a potassium salt and a calcium salt.

In another embodiment, the composition further comprises an acid activator. In one embodiment, the acid activator is citric acid. In some embodiments, the acid activator is present in an amount from about 0.1% to about 45% by weight. In some embodiments, the acid activator is present in an amount from about 0.5% to about 10% by weight. In some embodiments, the acid activator is present in an amount of about 3%, about 4%, or about 5% by weight. In some embodiments, the acid activator is in excess of the salt of chlorous acid. In some embodiments, the acid activator is about 1 to 10 times the concentration of the salt of chlorous acid. In embodiments where the composition comprises a clumping agent, it may be advantageous to use lower quantities of citric acid, for example, about 3%, about 4% or about 5% by weight, especially when the citric acid is applied as an aqueous solution, to avoid clumping of the composition prior to the use of the composition for pet waste management.

In some embodiments, the composition has a pH from about 2.5 to about 6. In another embodiment, the composition has a pH from about 2.5 to about 5.5, from about 2.5 to about 4.5, or from about 2.5 to about 3.5. In other embodiments, the composition has a pH below about 5, below about 4, or below about 3.5.

In some embodiments, the substrate comprises absorbent granules each granule comprising an absorbent core and a distinct layer surrounding the absorbent core.

In some embodiments, the absorbent granules comprise a clumping agent, wherein the clumping agent comprises bentonite, guar gum, starch, xantham gum, gum Arabic, gum acacia, silica gel, and mixtures thereof.

In some embodiments, the clumping agent comprises bentonite. In one embodiment, the bentonite is sodium bentonite.

In some embodiments, the composition comprises absorbent granules comprising an absorbent core, wherein the absorbent core comprises at least one of a clay, expanded perlite, quartz, feldspar, calcium bentonite, calcite, illite, calcium carbonate, carbon, mica, Georgia white clay, hectorite, zeolite, smectite, opal, kaolinite, pumice, tobermorite, slate, gypsum, vermiculite, halloysite, sepiolite, marl, diatomaceous earth, dolomite, attapulgite, montmorillonite, Monterey shale, Fuller's earth, silica, fossilized plant materials, perlites, perlite fines, and mixtures thereof.

In some embodiments, the composition further comprises an additive selected from the group consisting of a fragrance, an anti-microbial agent, an anti-sticking agent, an agent for controlling pH, a dye, a coloring agent, a de-dusting agent, a disinfectant, an additional odor control agent, and combinations thereof.

In some embodiments, the composition further comprises activated carbon.

In some embodiments, the composition has a density from about 50 kg/m³ to about 2000 kg/m³.

In some embodiments, the present disclosure comprises a composition comprising a clumping pet litter comprising a substrate with a specific surface area of less than about 60 m²/g and a salt of chlorous acid in an amount from about 0.1 to about 10% by weight. In some embodiments, the composition further comprises about 0.5% to about 50% by weight of an acid activating agent.

In another embodiment, the present disclosure comprises a deodorizer comprising a substrate with a specific surface area of less than about 60 m²/g and a salt of chlorous acid in an amount of about 0.1 to about 10% by weight. In some embodiments, the deodorizer further comprises about 0.5% to about 50% by weight of an acid activating agent.

In some embodiments, the present disclosure comprises a method of reducing malodor from animal waste comprising contacting the malodor with a composition comprising a substrate with a specific surface area of less than about 60 m2/g and a salt of a chlorous acid. In another embodiment, the present disclosure comprises a method of reducing malodor from animal waste comprising contacting the malodor with a composition comprising absorbent granules each granule comprising an absorbent core and a distinct layer surrounding the absorbent core, a salt of chlorous acid in an amount of about 0.1 to about 10% by weight. In some embodiments, the method further comprises about 0.5% to about 50% by weight of an acid activating agent, the % by weight being relative to the weight of the composition.

In an embodiment, the present disclosure comprises a method of reducing malodor from animal waste, the method comprising adding a deodorizer to a pet litter in a litter box, the pet litter having a different formulation than the deodorizer, the deodorizer comprising a composition for controlling the ammonia odor and other malodors in an animal litter, the composition comprising absorbent granules each granule comprising an absorbent core and a distinct layer surrounding the absorbent core, and a salt of chlorous acid in an amount of about 0.1 to about 10% by weight. In some embodiments, the method further comprises from about 0.1% to about 45% by weight of an acid activating agent, the % by weight being relative to the weight of the composition. In some embodiments, the acid activating agent is present in an amount from about 0.5% to about 10% by weight.

The compositions and methods of the present disclosure can comprise absorbent granules. Non-limiting examples of absorbent granules include non-swelling clay agglomerated into clay particles which are coated with clumping agent, such as a swelling clay. The non-swelling clay used in the agglomeration process can be about 0.3 millimeter (50 mesh) or smaller in size and is sometimes referred to as a clay seed base or a seed material. In an exemplary embodiment, clay particles range in size from about 0.03 mm to about 0.15 mm.

In an exemplary embodiment, the non-swelling clay can be agglomerated using a pin mixer. A swelling clay can be applied to the agglomerated particles to form a coating. Non-limiting examples of clumping agents include sodium bentonite powder and a bentonite/guar gum blended powder. In some embodiments, the coating may be further augmented with either or both of an odor control agent and an anti-microbial agent. In one embodiment, a salt of chlorous acid in an amount of about 0.1 to about 10% by weight is present in the coating. The coated particles or absorbent granules can be spherical in shape. The spherical shape is by way of example only, a host of shapes and sizes of coated particles can be produced by the embodiments and processes described herein.

In one specific embodiment the non-swelling clay can be sourced from recovery of waste fines which include calcium-montmorillonite. The calcium-montmorillonite fines can be agglomerated in a pin mixer using water as a binder. The agglomerated fines have a moisture content of about 20% to about 40%. In another embodiment, the fines have a moisture content of about 28% to about 34%. The agglomerated fines can then be coated with a bentonite powder with a particle size of about 0.15 mm or smaller using a centrifugal coater or a rotary coater/dryer system.

In one embodiment, the non-swelling clay is fed into a pin mixer using a screw extruder. Moisture (e.g. water) is added to the fines to act as a binder, in one embodiment about 28%, while in the extruder. The fines and the moisture result in a cake-like substance as it enters the pin mixer. A pin mixer includes a shaft with a series of pins that breaks up the cake and results in the formation of small, spherically shaped particles which are separated from the cake-like batch using shaker screens. As previously described, in one embodiment, the non-swelling clay is about 0.3 mm (50 mesh) or smaller in size and after addition of the moisture and the pin mixing process results in particles from about 0.3 mm to about 3 mm. Other methods are contemplated which include using binders of guar gum and water or starch and water.

Another embodiment utilizes a blend of non-swelling clay and bentonite with water as a binder to produce the particles through the pin mixing process. Still another embodiment utilizes sodium bentonite with water as a binder to produce particles from about 0.25 mm to about 3 mm in size through the pin mixing process. The agglomerated particles, including the clay and bentonite embodiment, or the bentonite embodiment, can then be coated with a bentonite powder of about 0.15 mm or smaller using a centrifugal coater or a rotary coater/dryer system for improved clumping capability.

In alternative embodiments, methods for coating an outer surface of non-swelling clay particles with a clumping agent include utilization of at least one of a fluidized bed dryer, a semi-continuous centrifugal coater or a rotary coating and drying system. In the rotary system, the clay particles and clumping agent are tumbled in a drum to mix for about 60 seconds. The litter is then removed from the drum and the drum is heated to about 300° F. to about 400° F. and the litter is returned to the drum and dried until about an 8% moisture content is obtained.

The resulting coated litter is typically in the 8 to 50 mesh size range, with a moisture content from about 15% to about 5%, preferably with a moisture content of about 8%. In one embodiment, the bentonite coating is about 20% to about 40% by weight of a coated particle. In an alternative embodiment, the bentonite coating is about 25% to about 35% by weight of a coated particle. In a further alternative embodiment, the bentonite coating is about 30% by weight of a coated particle.

In alternative method for producing the litter, the agglomerated fines are placed in a fluidized bed and bentonite coating is sprayed in a low concentration solution.

The litter resulting from the compositions and methods described above has superior clumping properties as the active clumping agent is kept on the surface of the particles, where the clumping bonds are formed. In addition, the litter has a dust content which is lower than known clumping litters, resulting in less tracking, as the coating processes described above result in a shell being formed around the agglomerated particles. Further, the litter is easier to remove from litter boxes than known clumping litters as the litter described herein is less likely to attach to litter boxes.

In the above described embodiments, coating with bentonite provides a litter which includes the clumping and absorption qualities of a litter which is composed essentially of sodium bentonite. However, due to the coating process, the amount by weight of sodium bentonite is reduced over known clumping litters, resulting in more efficient use of the sodium bentonite while providing a production cost savings over those litters with higher percentage amounts of sodium bentonite. In addition, the coated litter produced provides a lighter weight product and has a unique, homogeneous appearance that appeals to consumers. Further, the agglomeration process results in a utilization of clay product fines, which heretofore have been considered waste products, and since clay is not biodegradable, clay fines have traditionally required space for disposal.

In some embodiments, the composition comprises a substrate that is an absorbent granule having (i) a non-agglomerated particle comprising a perlite; and (ii) a coating on an outer surface of the particle, the coating comprising a clumping agent. In a particular embodiment, the clumping agent comprises bentonite.

In another embodiment, the composition comprises a substrate that is an absorbent granule having (i) a particle consisting essentially of expanded perlite; and (ii) a coating on an outer surface of the particle, the coating comprising a clumping agent. In a particular embodiment, the clumping agent comprises bentonite.

In some embodiments, the method for manufacturing the substrate involves (i) feeding perlite particles having a bulk density in the range of 25-300 kg/m³ into a coater; (ii) adding a liquid to the coater to create wet perlite particles; and (iii) feeding bentonite having a size range of about 0.15 mm or smaller into the coater to coat the wet perlite particles.

The substrates of the present disclosure include perlite particles coated with a clumping agent. In one particular embodiment, the particles are non-agglomerated particles comprising a perlite. In another particular embodiment, the particles consist essentially of expanded perlite. In yet another particular embodiment, granules of litter include an expanded perlite core coated with a mixture of sodium bentonite powder and guar gum.

Perlite is a generic term for a naturally occurring siliceous rock. One feature which sets perlite apart from other volcanic glasses is that when heated to a suitable point in its softening range, it expands from four to twenty times its original volume. This expansion is due, at least in part, to the presence of two to six percent combined water in the crude perlite rock. Firing, i.e., quickly heating to above 1600° F. (871° C.), causes the crude rock to pop in a manner similar to popcorn yielding a very open, highly porous structure referred to as expanded perlite.

Where expanded perlite is employed in the litter compositions, the bulk density of expanded perlite is typically in the range of 50 to 300 kg/m³. In one embodiment, for example, the bulk density of the expanded perlite of a coated litter of the invention is in the range from about 55 to about 80 kg/m³ (e.g., 55 kg/m³, 56 kg/m³, 58 kg/m³, 60 kg/m³, 62 kg/m³, 64 kg/m³, 66 kg/m³, 68 kg/m³, 70 kg/m³, 72 kg/m³, 74 kg/m³, 76 kg/m³, 78 kg/m³, or 80 kg/m³). In another embodiment, for example, the bulk density of the expanded perlite is in the range from about 55 to about 96 kg/m³ (e.g., 55 kg/m³, 56 kg/m³, 58 kg/m³, 60 kg/m³, 62 kg/m³, 64 kg/m³, 66 kg/m³, 68 kg/m³, 70 kg/m³, 72 kg/m³, 74 kg/m³, 76 kg/m³, 78 kg/m³, 80 kg/m³, 82 kg/m³, 84 kg/m³, 86 kg/m³, 88 kg/m³, or 90 kg/m³). In one particular embodiment, for example, the bulk density of the expanded perlite is approximately 72 kg/m³. In other particular embodiments, for example, the bulk density of the expanded perlite is approximately 120 kg/m³ or approximately 160 kg/m³.

Perlite can be further defined by its particle size. A range of particle sizes is preferred for the low density coated litters described herein. In one embodiment, the particle size of expanded perlite is in the range of U.S. sieve −8 to U.S. sieve +30. In another embodiment, the particle size of expanded perlite is in the range of U.S. sieve −6 to U.S. sieve +40. In some embodiments, the expanded perlite particles are not evenly distributed within the size range.

While typically at least some moisture is present in order to facilitate the coating process, the moisture content of the litter material described herein is relatively low. In one embodiment, for example, the moisture content (expressed as a percentage by weight) of the expanded perlite of the low density coated litter is between approximately 0% and 3%. In another embodiment, for example, the moisture content (expressed as a percentage by weight) is from about 2% and 3%. In yet another embodiment, the moisture content (expressed as a percentage by weight) is approximately 0.5%.

In some embodiments, the absorption of the expanded perlite particles is measured wt/wt from about 100% to about 800%, and measured by volume, is at least 20%. In one embodiment, the absorption of the expanded perlite particles, measured wt/wt is approximately 600% and, measured by volume, is approximately 45%.

The core perlite materials are coated with a clumping agent; i.e., an agent when wetted results in the binding of adjacent particles. Representative clumping agents include, for example, bentonite (such as sodium bentonite), guar gums, starches, xanthan gums, gum Arabic, gum acacia, silica gel, and other minerals, and mixtures a mixture thereof. In one embodiment, the clumping agent comprises bentonite.

In one embodiment, the clumping agent comprises sodium bentonite. Sodium bentonite is described in the industry as a “swelling” clay because particles of sodium bentonite enlarge in size and volume when they absorb moisture. In addition, sodium bentonite particles exhibit gel-like qualities when wet that promote clumping of the sodium bentonite particles when liquid (such as urine) is applied. In another embodiment, the clumping agent comprises a mixture of sodium bentonite and guar gum.

Where sodium bentonite is employed as or in the clumping agent, the bulk density of the bentonite is typically in the range of 600 to 1125 kg/m³ (e.g., 600 kg/m³, 700 kg/m³, 800 kg/m³, 900 kg/m³, 1000 kg/m³, or 1100 kg/m³). In one particular embodiment, for example, the bulk density of the sodium bentonite is approximately 1125 kg/m³ (approximately 70 lb/ft³).

In one embodiment, the moisture percentage of the sodium bentonite of the low density litter is from about 6% to about 7% (e.g., 6.1%, 6.2%, 6.3%, 6.4%, 6.5%, 6.6%, 6.7%, 6.8%, or 6.9%). In a particular embodiment, the moisture percentage of the sodium bentonite is approximately 6.24%.

The bentonite of the low density coated litter can be provided as a powder or “fines” with a size of 0.2 mm or smaller. In an exemplary embodiment, the size of the sodium bentonite particles is approximately 0.15 mm or smaller.

In general, methods for preparing litter compositions in accordance with the disclosure involve coating a perlite (and/or an expanded perlite) with a clumping agent. In one embodiment, perlite is screened to eliminate particles smaller than the range of particle sizes selected for the particular embodiment of litter. For example, expanded perlite may be screened to eliminate particles smaller than 50 U.S. sieve, more preferably smaller than approximately 40 U.S. sieve, still more preferably smaller than approximately 30 U.S. sieve. Commercially available shaker screens may be utilized.

The perlite particles can be placed in an enrobing machine to agitate the particles. This assists in the reduction of fines which, in turn, aids in dust abatement. In an exemplary embodiment, expanded perlite particles are weighed before or as they enter the enrober and the particles are sprayed with water. The amount of water added generally depends upon the weight of the expanded perlite particles included in the enrober. In one embodiment, for example, the weight of water added is from about 20 to about 90 percent of the weight of the expanded perlite particles (e.g., 30%, 40%, 50%, 60%, 70%, 80, or 90%). In another embodiment, for example, the weight of water added is from about 50 percent to about 85 percent of the weight of the expanded perlite particles (e.g., 50%, 55%, 60%, 65%, 70%, 75%, 80%, or 85%). In one particular embodiment, for example, the weight of water added is approximately 65 percent of the weight of the expanded perlite particles. Enrobing may also promote gelling of the bentonite coating material, as further described below.

The perlite particles can be coated with the clumping agent (e.g., sodium bentonite) in a coater. By way of example, centrifugal coating methods can be employed. For instance, a batch of perlite particles are metered onto a feed belt by volume and fed into the coater as it rotates. Perlite particles roll inside the chamber of the coater in the direction of rotation. In an optional preconditioning step, the perlite particles are spun in the coater for a period of time (e.g., 30 to 60 seconds) prior to coating.

Water can be added to the coater while the coater is spinning. Water added may be added based on the weight of the clumping agent to be added in the coater. The weight of water added is typically between approximately 10 to 100 percent of the weight of the clumping agent (e.g., 10%, 15%, 20%, 25%, 30%, 35%, 40%, 45%, 50%, 55%, 60%, 65%, 70%, 75%, 80%, 85%, 90%, 95%, or 100%). In one embodiment, for example, the weight of water added is between approximately 10 to 60 percent of the weight of the clumping agent (e.g., 10%, 15%, 20%, 25%, 30%, 35%, 40%, 45%, 50% 55%, or 60%). In another embodiment, for example, the weight of water added is between approximately 10 to 40 percent of the weight of the clumping agent (e.g., 10%, 15%, 20%, 25%, 30%, 35%, or 40%). In the alternative, water addition may occur in the enrober or in both the enrober and the coater.

The clumping agent (e.g., sodium bentonite) is metered into the coater. In general, the quantity of clumping agent added into the coater is based on the volume of perlite particles. In one embodiment, for example, from about 5 to about 45 pounds of sodium bentonite are added per cubic foot of expanded perlite (e.g., 5 pounds, 10 pounds, 12 pounds, 14 pounds, 20 pounds, 30 pounds, 35 pounds, 40 pounds, or 45 pounds). In another embodiment, for example, from about 20 to about 35 pounds of sodium bentonite are added per cubic foot of expanded perlite (e.g., 20 pounds, 25 pounds, 30 pounds, or 35 pounds). In yet another embodiment, from about 20 to about 30 pounds of sodium bentonite are added per cubic foot of expanded perlite (e.g., 20 pounds, 25 pounds, 27 pounds, 29 pounds, or 30 pounds). In one particular embodiment, approximately 30 pounds of sodium bentonite are added per cubic foot of expanded perlite.

Other coating materials, such as guar gum, may be included in the coater in addition to or in lieu of a bentonite-based clumping agent. Such materials may be added as a mixture, along with the bentonite, or they may be added in a separate step. As the bentonite (or other coating material) is metered into the chamber of the coater, it combines with the wet, spinning expanded perlite and forms a coating on the expanded perlite.

To achieve a more uniform coating, the coated perlite (e.g., expanded perlite coated with clumping agent) can be contacted (e.g., misted or sprayed) with additional water. In general, water added is added based on the weight of the clumping agent in the coater. In an exemplary embodiment, the weight of water added is from about 1 to about 5 percent of the weight of the clumping agent (e.g., 1%, 2%, 3%, 4%, or 5%). In another exemplary embodiment, the weight of water added is from about 5 to about 10 percent of the weight of the clumping agent (e.g., 5%, 6%, 7%, 8%, 9%, or 10%). In a further exemplary embodiment, the weight of water added is from about 1 to about 3 percent of the weight of the clumping agent (e.g., 1%, 2%, or 3%). In one particular embodiment, the weight of water added is approximately 2 percent of the weight of the clumping agent. In another particular embodiment, the weight of water added is approximately 5 percent of the weight of the clumping agent. In another particular embodiment, the weight of water added is approximately 9 percent of the weight of the clumping agent.

In an alternative embodiment, water may be added in a quantity appropriate to achieve a particular target moisture content following coating. In one embodiment, for example, water is added in a quantity appropriate to achieve a target moisture content from about 20 to about 40 percent (e.g., 20%, 21%, 22%, 23%, 24%, 25%, 26%, 27%, 28%, 29%, 30%, 31%, 32%, 33%, 34%, 35%, 36%, 37%, 38%, 39%, or 40%). In another embodiment, for example, water is added in a quantity appropriate to achieve a target moisture content from about 25 to about 30 percent. In one embodiment, a rotary system is utilized, where expanded perlite particles, bentonite, and water are tumbled in a drum.

The coated particles are transferred to a dryer. Drying removes moisture from the coated particle without substantially removing the coating or substantially damaging the finished product. A fluidized bed dryer is utilized in certain embodiments. Typically, the coated particles are dried to have a moisture content ranging from about 1.5% to about 20%. In one embodiment, for example, the coated particles are dried to a moisture content ranging from about 5% to about 15% (e.g., about 5%, about 7%, about 9%, about 11%, about 13%, or about 15%). In another embodiment, for example, the coated particles are dried to a moisture content ranging from about 7% to about 10% (e.g., about 7%, about 8%, about 9%, or about 10%). In one particular embodiment, for example, the final moisture content of the coated litter product is approximately 10%. In another particular embodiment, the coated particles are dried to a moisture level sufficient to achieve a relatively uniform appearance of the coated particles.

Another screening process can be used. A vibratory screener may be used to remove coated expanded perlite particles larger than a mesh size of about 8, and smaller than a mesh size of about 40. Any excess coated expanded perlite separated in the screening process may be, for example, ground and added to other litter products or used in other odor or moisture control products.

Various additives may be optionally applied to the coated litter product. Additives may include, for instance, an odor control agent(s), a fragrance(s), an anti-microbial agent(s), an anti-sticking agent(s), an agent(s) for controlling pH, a powder(s) for coloring, dyes, a coloring agent(s) and/or colored particles, a de-dusting agent(s), a disinfectant(s), or combinations thereof. In one embodiment, for example, at least a portion of the coated particles are further coated with a colorant. Various characteristics of coated litter products of the invention represent significant improvements over existing litter products.

By way of example, the density of coated litter compositions of the disclosure is relatively low, compared to other litter products. Typically, for example, the density of the coated litter product is from about 200 to about 600 kg/m³. In one embodiment, the density of the coated litter product is from about 300 to about 500 kg/m³ (e.g., 300 kg/m³, 350 kg/m³, 400 kg/m³, 450 kg/m³, or 500 kg/m³). In another embodiment, the density of the coated litter product is from about 350 to about 450 kg/m³ (e.g., 350 kg/m³, 400 kg/m³, 450 kg/m³). In one particular embodiment, the density of the coated litter product is approximately 350 kg/m³. In another particular embodiment, the density of the coated litter product is approximately 400 kg/m³. In another particular embodiment, the density of the coated litter product is approximately 450 kg/m³. Use of expanded perlite, for example, which is naturally lightweight, that is not agglomerated, crushed, extruded, or otherwise altered in a manner that increases its density, contributes to the desirable low density of the coated litter products of the invention and offers significant improvements over prior art litters. In one preferred embodiment, the perlite material is a non-agglomerated material; that is, it is not agglomerated or otherwise gathered into a mass or clustered with any other material.

In some embodiments, the perlite particles are substantially coated with the clumping agent. In one embodiment, for example, the particles are more than 75% coated. In other embodiments, for example, the particles are more than 85%, more than 95%, or more than 99% coated. In one embodiment, the coating material wholly surrounds or enrobes the particles. In some embodiments, agglomerated particles consisting mainly of expanded perlite can also be used.

Clumping litter consisting primarily of small, fine granules produces thin, large clumps when exposed to liquid, such as animal urine. On the other hand, clumping litter consisting primarily of larger granules produces columns of clumped litter. A range of various granule sizes produces a somewhat tortuous path for urine (or other liquid). The clumping litter material of the present disclosure includes, in various embodiments, perlite and/or expanded perlite particles that have a particular size distribution, as discussed herein. Accordingly, the perlite particles used to produce the coated litter product of the invention may not be agglomerated, crushed, extruded, or otherwise materially altered (other than to receive a coating), the end product retains the benefits of the size distribution. In other embodiments, the perlite particles may be agglomerated.

### EXAMPLES

### Example 1

Three different litter substrates were used and modified according to the procedures described herein. Litter substrate A comprised a clay core with a bentonite coating, litter substrate B comprised an expanded perlite core with a bentonite coating and litter substrate C comprised expanded perlite. The starting pH of each substrate was measured by placing a portion of the substrate in water (e.g. 2 g or 5 g of substrate in 50 g of water) and then measuring the pH using a pH meter. The approximate specific surface area, as determined using nitrogen adsorption at 77 Kelvin, and the starting pH of each substrate is shown in Table 1.

TABLE 1

| | Starting | Approx. Specific |
|---|---|---|
| Substrate | pH | Surface Area (m²/g) |
| Litter Substrate A | 9.2 | 60 |
| Litter Substrate B | 10.8 | 18 |
| Litter Substrate C | 8.5 | 1 |

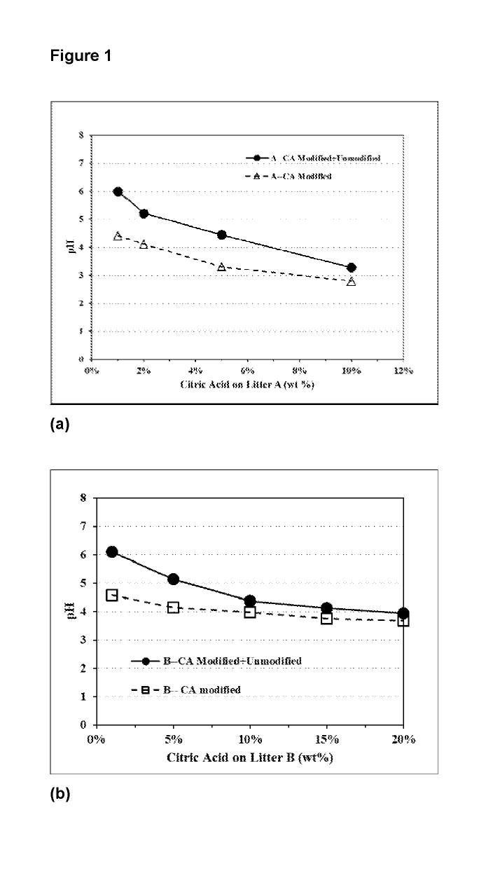

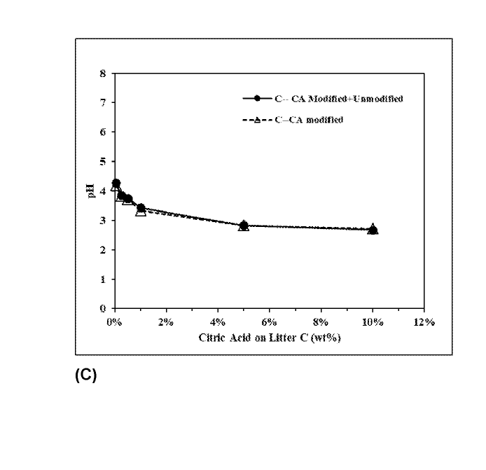

In embodiments utilizing both a chlorite salt and an acid activator, the final composition should be in the acidic range (e.g. pH≤6). To determine the pH of citric acid modified substrates, several modified substrate samples were prepared by treating each substrate with an aqueous solution of citric acid and then drying. Additionally, a portion of each citric acid modified substrate was mixed (after drying) with an equal amount of unmodified substrate. For example, 10 g of citric acid modified Litter Substrate A was mixed with 10 g of unmodified Litter Substrate A. The pH of the citric acid modified substrate and the pH of the mixture of modified and unmodified substrates was measured by adding roughly the same volume of litter samples (e.g. 2 g, 5 g, or 10 g) to 50 g of water and measuring the pH using a calibrated pH meter. The results of the measurements are shown in FIG. 1 (a), (b) and (c).

The amount of citric acid required to lower the pH of the substrate is believed to be, at least in part, a function of the starting pH and surface area. In addition, the effect of the substrate matrix, such as surface area and pH, is evident when comparing the pH of a composition containing only the citric acid modified litter substrate to the pH of the composition upon addition of unmodified litter substrate of the same type. For example, with litter substrate C, no appreciable change in pH is measured upon combination of the citric modified litter substrate C with an equal amount of unmodified litter substrate C, compared to a change in pH for both litter substrate A and B upon addition of unmodified litter substrate.

### Example 2

Compositions comprising sodium chlorite and citric acid were prepared according to the following general procedure. Sodium chlorite was dissolved in water and a portion of substrate was treated with the aqueous sodium chlorite solution. For samples modified with 5 wt % citric acid, citric acid was dissolved in water and a separate portion of substrate was treated with the aqueous citric acid solution. Each portion of treated substrate was separately dried overnight in air at room temperature. The samples modified with a higher level of citric acid (e.g. 20 wt %, 40 wt %) were prepared by dry mixing citric acid powder with litter substrate, and then heating the mixture in oven at 160° C. for 2 hrs. Equal portions of dried substrate containing citric acid and dried substrate containing sodium chlorite were mixed together for subsequent testing. A list of prepared compositions is shown in Table 2.

TABLE 2

| Litter Substrate | Citric Acid | Sodium Chlorite |
|---|---|---|
| A | 5 wt. % | 0.5 wt. % |
| A | 20 wt. % | 1 wt. % |
| B | 5 wt. % | 1 wt. % |
| B | 20 wt. % | 1 wt. % |
| B | 40 wt. % | 1 wt. % |
| B | 5 wt. % | 5 wt. % |
| B | 20 wt. % | 5 wt. % |
| B | 40 wt. % | 5 wt. % |

The ability of each composition to generate ClO₂ was measured using the following general procedure. A composition containing equal portions of dried substrate treated with citric acid and dried substrate treated with sodium chlorite was placed in a sealed container with a valve that was connected to a Draeger testing tube. The container was briefly opened and 20 mL of water was added. The container was immediately closed and a measurement was performed by drawing 100 mL of air from the container using a hand pump. The concentration of ClO₂ in parts per million (ppm) was read to the best possible precision based on the color change in the Draeger tube.

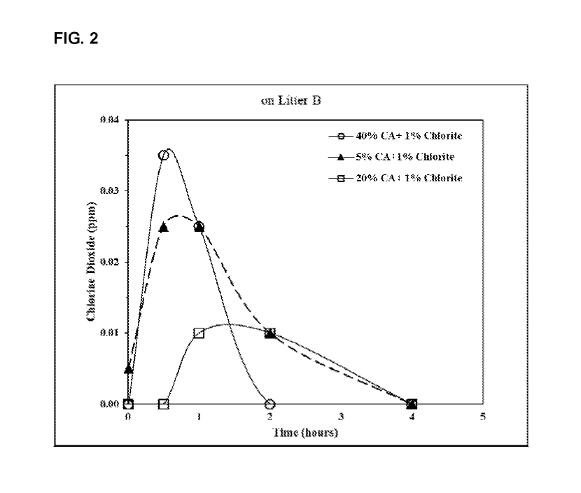

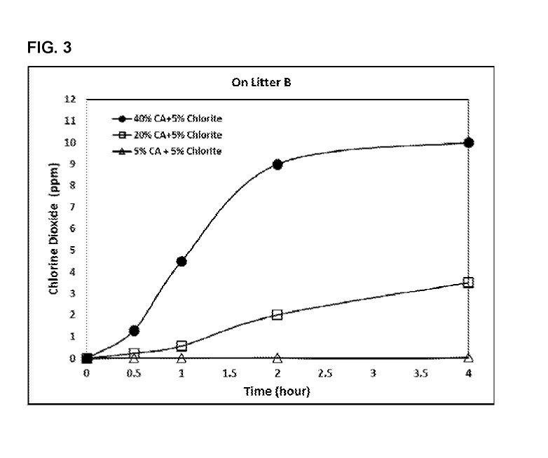

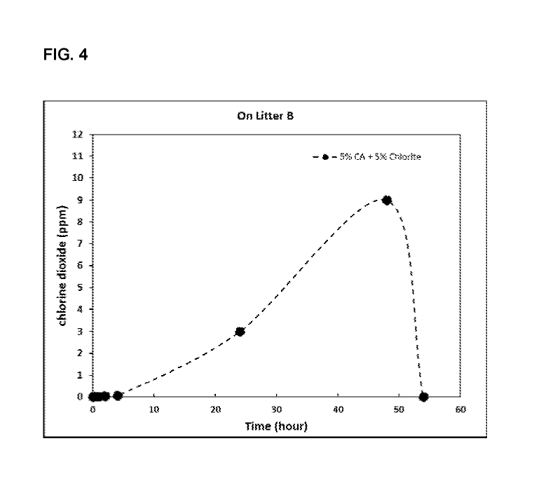

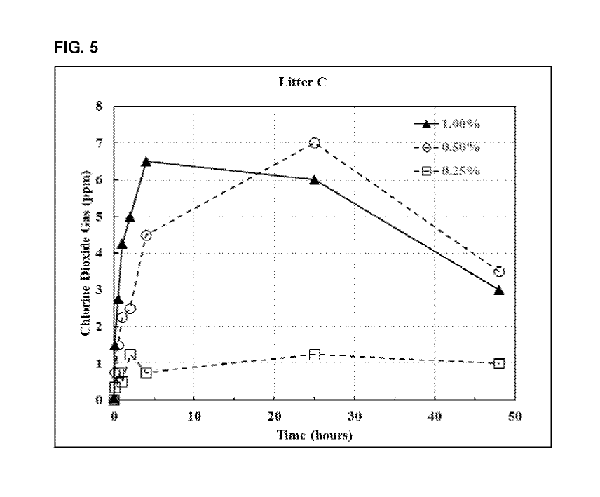

The amount of ClO₂ generated upon wetting of litter substrate A was below detectable limits for the composition containing 5 wt. % citric acid, as well as for the composition containing 20 wt. % citric acid. The amount of ClO₂ generated over time upon wetting of litter substrate B as a function of citric acid concentration at a constant concentration of sodium chlorite (1%) is shown in FIG. 2. The amount of ClO₂ generated over time upon wetting of litter substrate B as a function of citric acid concentration at a constant concentration of sodium chlorite (5%) is shown in FIG. 3 and FIG. 4. The amount of ClO₂ generated over time upon wetting of litter substrate C at a fixed citric acid concentration (0.5%) and varying sodium chlorite concentration is shown in FIG. 5.

Without being bound by a particular theory, it is believed that the high absorptive capacity of litter substrate A may result in an immediate and near complete absorption of any ClO₂ generated, thereby resulting in no measurable quantities of the ClO₂ in the environment above or around the substrate.

Litter substrate B has a moderate surface area, therefore some generation of ClO₂ was measured. The amount of ClO₂ generated directly correlated with the amount of citric acid on the substrate. The concentration of ClO₂ generated with 1% of sodium chlorite and varying amounts of citric acid on litter substrate B reached a maximum between about 30 minutes to about 1 hour while the concentration of ClO₂ generated with 5% sodium chlorite and 5% citric acid on litter substrate B reached a maximum around 48 hours.

Litter substrate C has the lowest surface area of the substrates tested and provides an ideal substrate for ClO₂ generation. Without being bound by a particular theory, it is believed that the expanded pore structure of the perlite on a micrometer or submicrometer scale provides sufficient volume to hold the chlorite compound and acid activator, while the low surface area does not readily absorb the ClO₂ gas generated, thus making it available for deodorizing and disinfecting the composition and close environment. Further, with litter substrate C, when the concentration of citric acid and sodium chlorite in the composition were equal at 0.5% the peak concentration of ClO₂ was seen at about 24 hours. The rate of ClO₂ generation also appears to be a function of chlorite concentration under these conditions. When the chlorite is the limiting reagent (e.g. 0.25 wt. % sodium chlorite and 0.5 wt. % citric acid) the ClO₂ generation is just a little over 1 part per million (ppm).

### Example 3

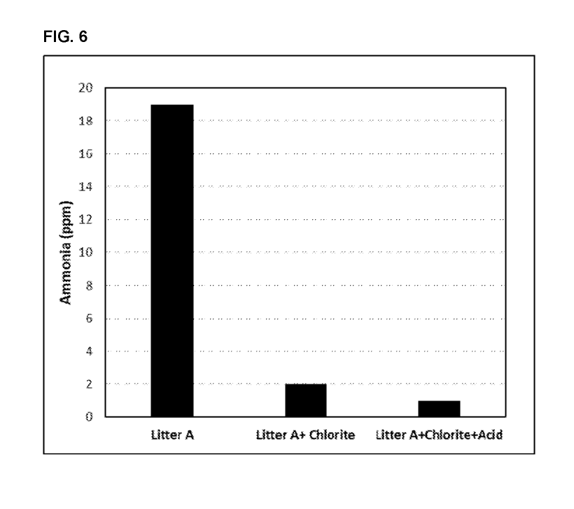

Experiments were conducted to determine the effectiveness of compositions containing chlorite compounds on ammonia control. Three clay-based litter compositions were prepared as shown in Table 3 below. Ammonia concentration was measured using Draeger ammonia testing tubes according to the following procedure. Synthetic urine (20 mL) was mixed with 0.1 mL of urease (U1875 from Sigma Aldrich) for 5 minutes. The liquid mixture was then poured into 200 grams of an unmodified or modified litter sample in a jar within a larger container. The larger container, connected to a Draeger ammonia tube via a valve, was closed as was the valve. After 24 hours the valve was opened and the 100 mL of gas was drawn from the container through the Draeger tube (100 ppm range). The valve was closed again. The ammonia concentration in parts per million (ppm) was determined to the best possible precision based on color change in the Draeger tube. The results are shown in FIG. 6.

TABLE 3

| Substrate | Modification |
|---|---|
| Clay-based litter A | Control sample |
| Clay-based litter A | Sodium chlorite (0.5 wt. %) treated and |
| | dried granules |
| Clay-based litter A | Mixture of sodium chlorite (0.5 wt. %) |
| | treated and dried granules + citric |
| | acid (0.5 wt. %) treated and |
| | dried granules |

Even in the absence of citric acid, chlorite is effective in reducing or stopping ammonia generation. Ammonia concentration produced from litter A is much higher in the absence of a chlorite compound. Without being bound by a particular theory, it is believed that the chlorite compound inhibits the urease enzyme and thus prevents the formation of ammonia.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A composition for controlling malodor of animal waste, comprising a substrate with a specific surface area of less than about 60 m²/g and a salt of chlorous acid in an amount from about 0.1% to about 10% by weight.

2. The composition of claim 1, wherein the salt of chlorous acid is selected from the group consisting of a sodium salt, a magnesium salt, a potassium salt and a calcium salt.

3. The composition of claim 1, further comprising an acid activator.

4. The composition of claim 3, wherein the acid activator is citric acid.

5. The composition of claim 3, wherein the composition has a pH from about 2.5 to about 6.

6. The composition of claim 3, wherein the acid activator is in excess of the salt of chlorous acid.

7. The composition of claim 3, wherein the acid activator is present in an amount from about 0.1% to about 45% by weight.

8. The composition of claim 1, wherein the substrate comprises absorbent granules each granule comprising an absorbent core and a distinct layer surrounding the absorbent core.

9. The composition of claim 8, wherein the distinct layer surrounding the absorbent core comprises a clumping agent.

10. The composition of claim 9, wherein the clumping agent comprises sodium bentonite.

11. The composition of claim 8, wherein the absorbent core comprises at least one of a clay, expanded perlite, quartz, feldspar, calcium bentonite, calcite, illite, calcium carbonate, carbon, mica, Georgia white clay, hectorite, zeolite, smectite, opal, kaolinite, pumice, tobermorite, slate, gypsum, vermiculite, halloysite, sepiolite, marl, diatomaceous earth, dolomite, attapulgite, montmorillonite, Monterey shale, Fuller's earth, silica, fossilized plant materials, perlite, perlite fines, and mixtures thereof.

12. The composition of claim 1, further comprising an additive selected from the group consisting of a fragrance, an anti-microbial agent, an anti-sticking agent, an agent for controlling pH, a dye, a coloring agent, a de-dusting agent, a disinfectant, an additional odor control agent, and combinations thereof.

13. The composition of claim 1, wherein the composition has a density from about 50 kg/m³ to about 2000 kg/m³.

14. A clumping pet litter comprising the composition of claim 1.

15. A deodorizer comprising the composition of claim 1.

16. A method of reducing malodor from animal waste comprising contacting the malodor with a composition comprising a substrate with a specific surface area of less than about 60 m²/g and a salt of chlorous acid in an amount from about 0.1% to about 10% by weight.

17. A method of reducing malodor from animal waste, the method comprising adding a deodorizer to a pet litter in a litter box, the pet litter having a different formulation than the deodorizer, the deodorizer comprising a substrate with a specific surface area of less than about 60 m²/g and a salt of chlorous acid in an amount from about 0.1% to about 10% by weight.

18. The method of claim 17, wherein the substrate comprises absorbent granules each granule comprising an absorbent core and a distinct layer surrounding the absorbent core.
